(19) United States
(12) Patent Application Publication
(10) Pub. No.: US 2019/0122757 A1
(43) Pub. Date: Apr. 25, 2019

(54) METHOD AND DEVICE FOR SOFTWARE-DEFINED THERAPY

(71) Applicant: Rui Lin, San Diego, CA (US)

(72) Inventor: Rui Lin, San Diego, CA (US)

(21) Appl. No.: 16/150,529

(22) Filed: Oct. 3, 2018

Related U.S. Application Data
(60) Provisional application No. 62/575,461, filed on Oct. 22, 2017.

(51) Int. Cl. G16H 20/70; G06F 21/60; G06F 8/34; G06F 11/36; G06F 8/60

(52) U.S. Cl. CPC G16H 20/70; G06F 21/602; G06F 8/65; G06F 11/3688; G06F 8/60; G06F 8/34

(57) ABSTRACT

The disclosure provides methods and apparatuses to make professional therapy programmable, accessible, and executable without the physical present of professionals. An embodiment is resided and distributed in three key subsystems, i.e., cloud, smartphone app, and smart medical device, and the supporting tool and the development environment. The smart device is equipped with the capabilities for reprogramming, for storage of programs, for control, and for the execution of the programs. The supporting tool and development environment are designed and provided to convert professional therapy into program file. The program file can be published, updated, discovered, and synchronized in the cloud. The smartphone app is designed and provided as a portal for user to discover, access, use, and run the therapy. The therapy can be controlled either by the smartphone app or the smart medical device. An end-to-end encryption method is designed to make sure the therapy is protected and secure.

### CROSS REFERENCE TO RELATED APPLICATIONS

This application claims the priority benefit under 35 U.S.C. § 119(e) to U.S. Provisional Patent Application Ser. No. 62/575,461, titled “METHOD AND APPARATUS FOR SOFTWARE-DEFINED THERAPY,” filed on Oct. 22, 2017, the disclosure of which is hereby incorporated by reference in its entirety for all purposes.

### FIELD OF THE INVENTION

The present invention is generally related to healthcare and more particularly, to systems, methods, and devices for software-defined healthcare therapy.

### BACKGROUND OF THE INVENTION

Technically, all organs and functions are regulated through brain and nervous system; a circuit of neurons communicating through neural impulses. Even endocrine system is under control of central nervous system by a complex array of feed-back mechanisms. Electrotherapy or Electroceuticals are a new category of therapeutic agents which act by targeting the neural circuits of organs or muscles to treat various conditions. The therapy involves mapping the neural circuitry and delivering neural impulses to these specific targets. In other words, the neural impulses that control the body will be entrained to regain the lost function and reestablish a healthy balance. Thus, they could regulate a host of bodily activities; food intake, cardiac activity, pancreatic activity, liver, kidney or spleen functions. They could even control inflammation and set right many pathologies like diabetes mellitus, obesity, hypertension, heart failure, cerebral-vascular and pulmonary diseases. It is estimated that electroceuticals will become a mainstay of medical treatment over the next two decades, benefiting up to 2 billion people—a quarter of the global population—who are suffering from chronic diseases.

Traditional Chinese Medicine (TCM) has been practiced for more than 2,000 years to relieve pain from various problems or health conditions and to improve overall health. TCM divides the body into 12 major anatomical sections called meridians or channels Simply speaking, a meridian or channel refers to a grouping of certain blood vessels, nerves and muscles. Each of these meridians also includes associated acupuncture points (acupoints). TCM wellness techniques are built on the theory of a meridian network, or a path in the human body through which the life-energy known as “qi” flows. If the path is blocked, one will experience sickness and pain.

Electrical Muscle Stimulation (EMS) and Transcutaneous Electrical Nerve Stimulation (TENS) have been used in bioelectronic devices to simulate various neural circuits for improved healing. EMS, first used by Egyptians 2,000 years ago, is employed to relax, enhance, and reshape muscles for stress release, for fast twitch response, endurance, strength, and more. TENS, first used in ancient Rome, is employed to stimulate the nervous system's ability to heal and release endorphins for pain-relief.

Actually, there are more known electrotherapy techniques, for example, VNS (Vagus Nerve Stimulation), nVNS (non-invasive Vagus Nerve Stimulation), CES (Cranial Electrotherapy Stimulation), and neuromodulation besides TENS, EMS, acupuncture. However, these treatments are not readily accessible to the average layperson. Professional health care providers who use these techniques to perform therapy on patients through the use of a medical device, e.g. EMS device or TENS unit. However, the medical device itself cannot perform the desired therapy automatically but needs to be operated by a provider, i.e. a healthcare professional with different kinds of neuromodulation waveforms or combination of complex waveforms for the desired efficacy. In addition, conventional home therapy devices only have a few basic therapy patterns with very limited effects and offer no capability to re-format the right waveforms for the right conditions in the right time to the average layperson.

### SUMMARY OF THE INVENTION

The present invention provides a system, a method, and a device for making professional therapy programmable, accessible, and executable without the physical presence of healthcare professionals. In one embodiment, the system includes three subsystems, i.e. the cloud, a smartphone app, and a smart medical device, along with a supporting tool and a development environment. The smart medical device can be equipped with the capabilities for reprogramming, storing of programs, controlling and executing the programs. The supporting tool and development environment can be designed and provided to convert professional therapy into a program file. The program file can be published, updated, discovered, and synchronized in the cloud. The smartphone app can be designed and provided as a portal for users to discover, access, use, and run the therapy on their smartphone device and the like. The therapy can be controlled either by the smartphone app or the smart medical device. An end-to-end encryption method can be designed to make sure the therapy is protected and secured.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, size, or applicability of the disclosure. It should be noted that for clarity and ease of illustration these drawings are not necessarily made to scale.

FIG. 1 illustrates an embodiment of a system architecture for software-defined programmable healthcare therapy;

FIG. 2 illustrates an embodiment for a process of healthcare therapy program creation, publication, and use;

FIGS. 3A-C illustrate several embodiments for synchronization between a smartphone device App and a smart medical device;

FIG. 4 illustrates an embodiment of a method to support off-line therapy and synchronization between the cloud, the smartphone App, and the smart medical device;

FIG. 5 illustrates an embodiment of a method for users to create their own therapy program; and

FIG. 6 illustrates an embodiment of a hardware block diagram for software-defined therapy.

### DETAILED DESCRIPTION OF EXEMPLARY EMBODIMENTS

The following description is presented to enable a person of ordinary skill in the art to make and use embodiments described herein. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the disclosure. The word “exemplary” is used herein to mean “serving as an example illustration.” Any aspect or design described herein as “exemplary” is not necessarily to be construed as preferred or advantageous over other aspects or designs. Thus, the present disclosure is not intended to be limited to the examples described herein and shown but is to be accorded the scope consistent with the claims.

The disclosure provides a common platform of a cloud, a smartphone App, and a smart medical device to make healthcare treatment procedures programmable and automatically operable with minimum manual operations involved. The systems, methods, devices and principles for software-defined therapy can be based on electricity, magnetics, light and the like, or any combination thereof. The efficacy and safety of the therapy depends on a complete treatment procedure including a number of important components such as waveforms, intensity, length of treatment time, place on the human body to apply the treatment, how many times a day, how long in terms of days, and the like. Thus, a complete healthcare therapy can be performed in terms of waveforms, initial intensity, time to apply the waveform, how long a waveform should be applied, change of waveform, and the like, can be defined by proprietary script instructions of a program and driven by the program. Intensity can be controlled by user to suit individual personalization.

Furthermore, in addition to the disclosed static nature of the pre-defined program there is a dynamic method to alter the waveform based on the artificial-intelligently analysis of the user usage pattern to give user the needed energy for therapy within the safety limit. For example, if user has selected the intensity level to the highest level and still pressing for more and if there is more headroom for safety we will increase, e.g., the pulse width to allow more energy for user.

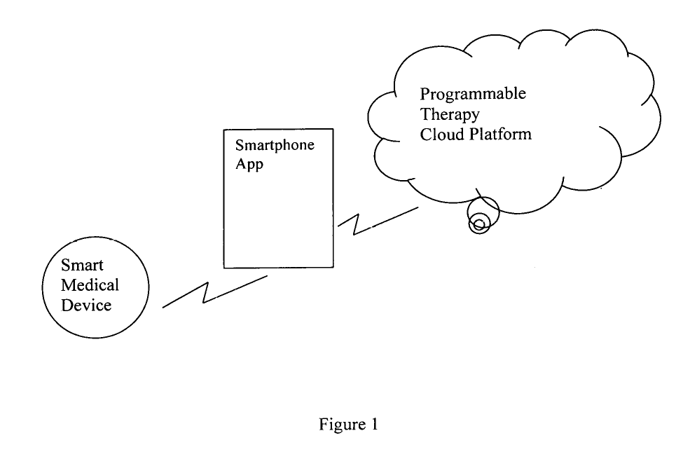

FIG. 1 illustrates an embodiment of a system architecture for software-defined programmable healthcare therapy. The system architecture includes programmable therapy stored in the cloud that can be accessed through a smartphone. The system architecture also includes an App on a smartphone device, and a corresponding smart medical device, such as a smart EMS device or smart TENS unit device and the like. Using this system, the latest therapy programs can be stored and maintained in the cloud. Users can access the desired therapy through the smartphone App and perform the desired therapy through the smart medical device.

As shown in this figure, the system architecture includes three subsystems: (1) a cloud service platform; (2) a smartphone App on a smartphone device; and (3) a smart medical device.

In one embodiment, the cloud service platform includes the following key modules: a user account management module, a user data module, a therapy program encryption module, and a management module, an operation management module, an App management module, a smart medical device firmware management module, a data mining module, a service management module, and the like.

In an embodiment, the smartphone App includes the following key modules: a synchronization with cloud on therapy program management module, an account information module, a user data module, a device firmware module, and the like. In an embodiment, the App provides a user interface and functions, for example, as a self-made therapy program, a therapy search and selection program, a therapy instruction program, a professional guide program, a therapy operation and control program, and the like, for the user to operate. The smartphone App can also interact with a smart medical device for link encryption and management, therapy management and control.

In an embodiment, the smart medical device can interact with the smartphone App for therapy control and management. It can also perform therapy program interpretation and execution.

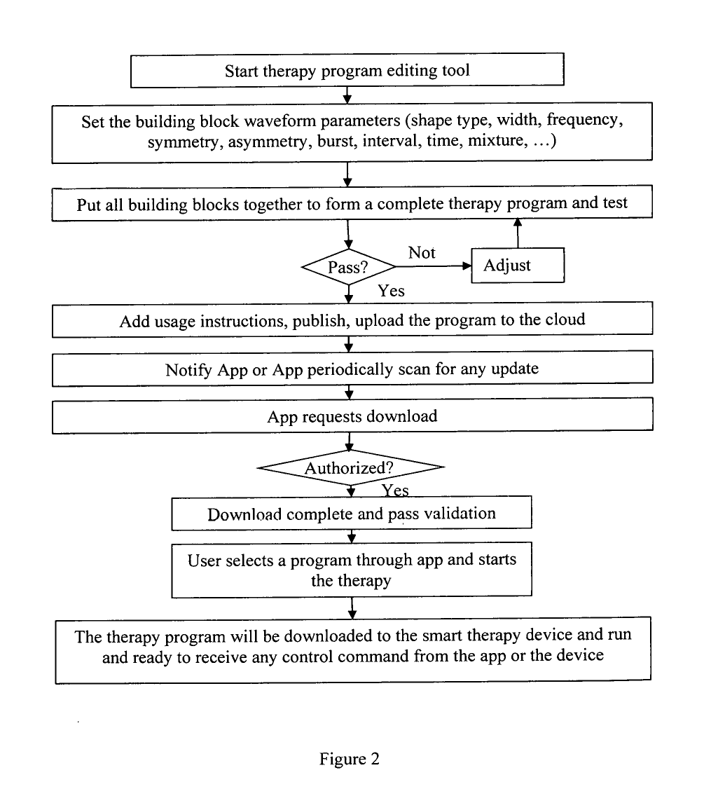

FIG. 2 illustrates an embodiment for a process of healthcare therapy program creation, publication, and use. As shown in this figure, a therapy program editing tool can be started and can be used to set the building block waveform parameters, e.g. shape type, frequency, symmetry, asymmetry, burst, interval, time, mixture, and the like. All of the building block waveform parameters can then be put together to form a complete therapy program. The program can then be tested and adjusted if needed. Once the program passes the test, usage instructions can then be added to the program, and published and/or uploaded to the cloud. The smartphone device App can then be notified of any new programs. In addition, the App can periodically scan the cloud for any new programs or updates.

In practice, the smartphone device App can request a download of one or more therapy programs, which can be checked to for authorization. Once the download is completed and the program passes validation, the user can select a desired therapy program through the App and can start the desired therapy. Alternatively, the therapy program can be downloaded directly to the smart medical device and ran. In addition, the smart medical device can receive any control command from the smartphone App or device.

In an embodiment, the therapy program editing tool allows professional healthcare providers to generate therapy programs. The tool provides a user interface (UI) for the user to input the characteristic parameters of the waveform, when it will be used, when it will be changed, changed characteristics, repetitive setting, and more. All the inputs can be translated into a serial of control commands and put into a program file.

The programmable waveforms, intensity, and the treatment running time can be supported in the common hardware in the smart medical device. The waveforms and running time are usually contained in the program file. The intensity for manual control can be provided to suit each individual's situation. The other important support functions such as program interpretation logics, link encryption logics, and the executable and control logics can also be included in the device.

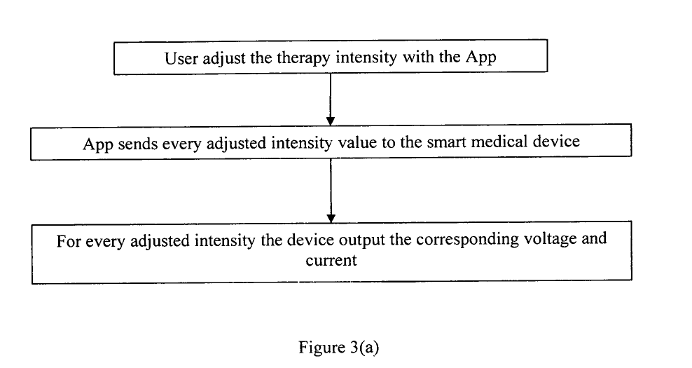

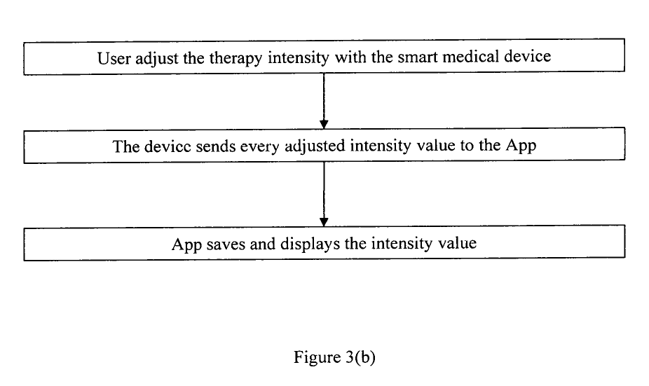

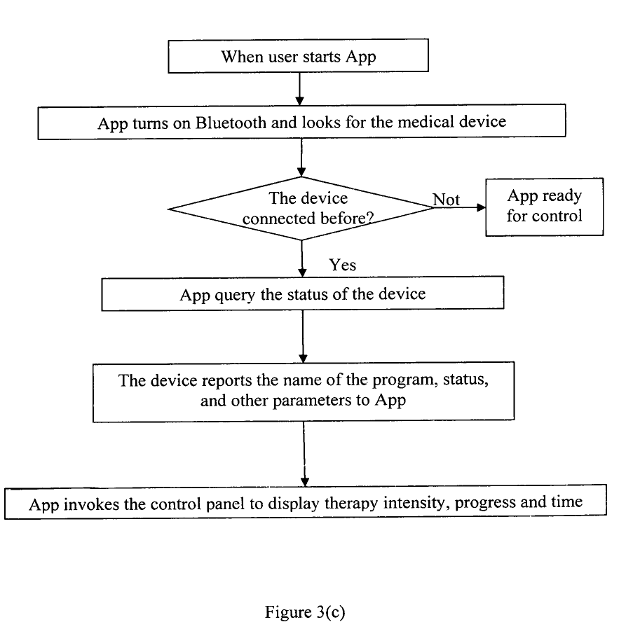

FIGS. 3A-C illustrate several embodiments for synchronization between a smartphone device App and a smart medical device.

As shown in FIG. 3A, a user can adjust the intensity of the therapy with the smartphone device App. The App can then send every adjusted intensity value to the smart medical device. Thus, for every adjusted intensity value, the smart medical device outputs the corresponding intensity, for example, in terms of voltage and current applied.

As shown in FIG. 3B, the user can adjust the intensity of the therapy with the smart medical device. The smart medical device can send every adjusted intensity value to the App. on the smartphone device; and the App can save and display the adjusted intensity value to the user.

As shown in FIG. 3C, when the user starts the App on the smartphone device, the App can turn on a wireless network such as Bluetooth® and the like, to look for the smart medical device. If the App and the smart medical device have been connected before, the App is then ready for control over the device. If the App and the smart medical device haven't been connected before, the App queries the status of the smart medical device; and the smart medical device reports the name of the program, status, and other parameters to the App. The App can then invoke the control panel on the smart medical device to display the therapy intensity, progress and time.

In an embodiment, the smartphone App can be used with one or more RF transceivers in order to securely bind and control multiple smart medical devices. The App and the smart medical device can maintain a secure database after successful binding. The next time when the smart medical device is turned on, the App can periodically search and exchange the database with the smart medical device. When the match is validated the App and the smart medical device can be automatically synchronized and bonded without user's involvement.

In another embodiment, a user can first use the App on their smartphone device to pre-securely bind with every device they have. A list of the bonded devices can be maintained by the App. The user can use the App to select any pre-bonded device to operate as long as they turn on the device. This way, the user can use one App to control multiple devices for therapy for multiple locations on the body. This eliminates the use of multiple transceivers in a controller to control multiple smart medical devices and saves cost.

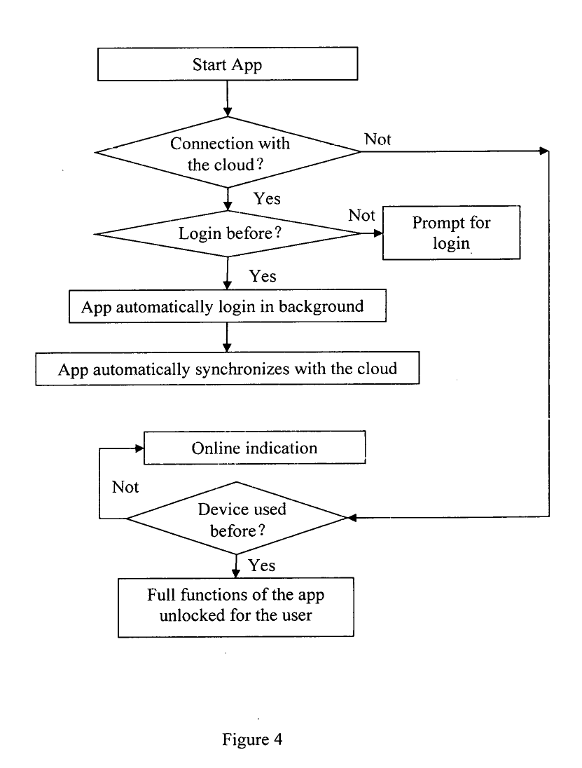

FIG. 4 illustrates an embodiment of a method to support off-line therapy and synchronization between the cloud, the smartphone App, and the smart medical device.

In one embodiment, the App on the smartphone device can be started and checked to see if it is connected with the cloud. If not, a query is made to see if the device has been used before. If not, an online indication is provided; and if yes, then the full functions of the App are unlocked for the user.

If the App is connected with the cloud, a query is made to see if the App has login before. If not, a prompt for the login is made. If yes, the App automatically logins in the background, and the App automatically synchronizes with the cloud.

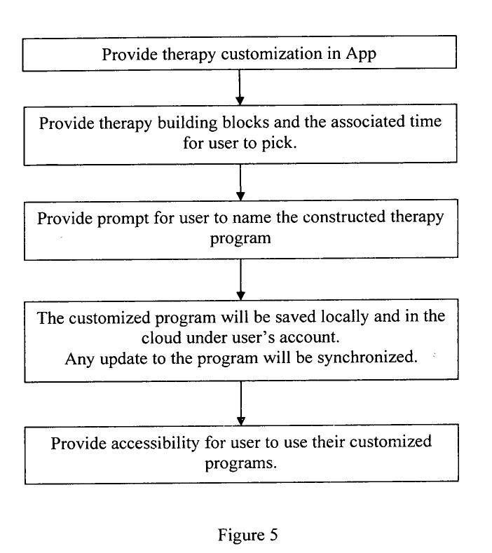

FIG. 5 illustrates an embodiment of a method for users to create their own therapy program. As shown in this figure, therapy customization can be provided in the smartphone device App. The user can then pick the provided therapy building blocks and the associated time for their use. Next, the user can be provided with a prompt for the user to name the constructed therapy program. The customized therapy program can then be saved locally and in the cloud under the user's account. Any future updates to the program can be synchronized with the program. Finally, accessibility can be provided to the user so they can use their customized programs.

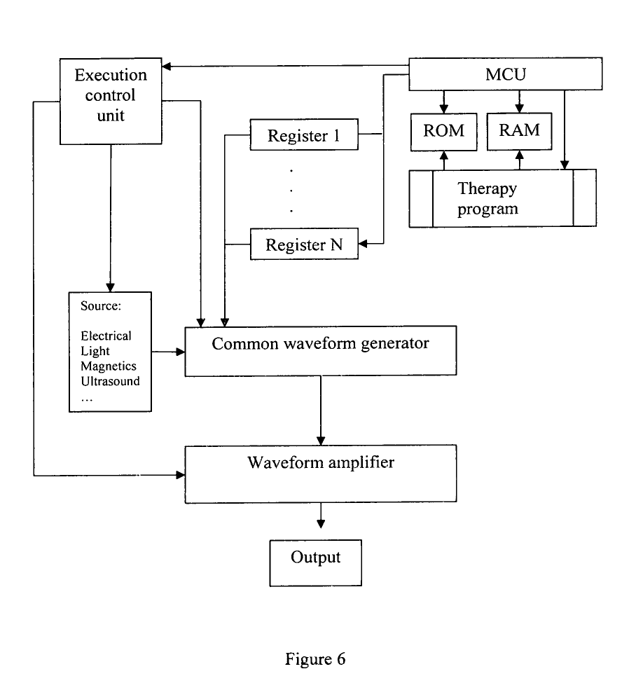

FIG. 6 illustrates an embodiment of a hardware block diagram for software-defined therapy. As shown in this figure, a hardware block diagram of the common waveform generator for software-defined therapy includes a microcontroller unit (MCU), which is the central control unit for generation of the therapy waveform and the execution of the therapy program.

In one embodiment, the MCU can receive the therapy program from the smartphone App via an encrypted link, and can then store it in a read only memory (ROM).

When receiving an operation command from the smartphone App or input from the device operation, the MCU can read the therapy program from the ROM and load it into a random-access memory (RAM), to interpret every control command of the program and execute it accordingly.

To execute the control commands of waveform generation, the MCU can load the corresponding waveform characteristic parameters into various control registers. Each control register controls different aspect of the waveform characteristics. After all control registers have been correctly setup, the right waveform is ready for an execution control unit to start it.

The execution control can be driven by the therapy program and commanded by the MCU state machine. It can select the correct input source(s) of the waveform, control the generation of the waveform at the right level or intensity and at the right time. The right intensity is setup by controlling the waveform amplifier.

A complete healthcare therapy procedure can be made from a therapy program and the associated instructions for its use. The instructions contain important information such as an explanation of the therapy method and the mechanism, pictures to show the place on the human body to apply the treatment, how many times a day, how long in terms of day, precautions, and the like.

A smartphone App offers the best way for users to find, read, and access to software-defined therapy services due to its popularity as an always carried tool, the screen, smart capability, and always networked. A smartphone App can be used to act as: (1) a bridge to link the cloud-based software-defined therapy with the smart medical; (2) an easy interacted portal for therapy; (3) a replacement of the screen for the smart medical; (4) a convenient way of remote control for therapy; (5) access to new therapies.

Healthcare therapy procedures can be created and stored in a therapy database in the cloud. A copy of the database can also be duplicated in the smartphone App for performance considerations. All records created and updated in the database can be time stamped. Whenever there is a new therapy procedure or an update of a procedure, the database can be updated in the cloud. A synchronization mechanism via notification or polling in the smartphone App can notify the user of the update, automatically initiate the download, and update the local database.

A set of control commands can be created to define a healthcare therapy program. The commands characterize the waveform used, to control when it is used, characteristic changes, and the time of changes. The waveform can be characterized in terms of type (e.g., sinusoid, square, triangular, sawtooth, width, symmetry, asymmetry), frequency, burst, and the like.

The various usage scenarios for the healthcare therapy procedures include but are not limited to the following: when a user selects a therapy procedure in their smartphone App, the user can first read and follow the instructions to get ready before the therapy. After the user starts the therapy, the corresponding program can be extracted and downloaded to the smart medical device. The smart medical device can start a state machine to open the program file to read, interpret the control commands, and execute the corresponding actions. In the mean-time, the smart medical device can receive the intensity control command from the smartphone App or from the smart medical device's intensity adjustment buttons to set the corresponding intensity. After the last control commands are read and executed, the therapy program can be stopped.

An initial set of programs have been developed to address pains/conditions, muscle and body building, and relaxation. These include but are not limited to a TENS (Transcutaneous Electrical Nerve Stimulation) and EMS (Electrical Muscle Stimulation) waveform.

Thus, the disclosure provides a method based on a pain profile of a user (pain level, pain frequency, pain duration, pain location, and the like). The smartphone App can recommend a list of programs for therapy based on medical expert system built from scientific research and care practice. For example, for joint pain, generic/advanced pain relief programs, nature stimulated endorphin program, generic/advanced muscle strengthen program, thorough relaxation program, and the like can be recommended.

Various healthcare therapy programs can be published and made available in the cloud can be marked as public or private to an individual. Therapy programs can be marked as free or paid. Public therapy programs let millions of user's access to the latest and greatest therapy instantly and have the consistent quality treatment. Private therapy programs let professionals customize their treatment to better suit individuals. Researchers can use the feature of private therapy program to develop and test their newly discovered therapy techniques before making them available to pubic.

As described herein, a specific data collection and mining method for software-defined healthcare therapy has been developed. A usage data record for each user can be established including: the program used, the time of use, the length of use. If the smart medical device is used offline, i.e., without the App on the smartphone, the data can be collected and kept in the smart medical device. As soon as the smart medical device can be connected to the App on the smartphone device, the stored usage data can be transmitted to the App and then to the cloud. Usage data can be mined to find out important information such as a user's condition or purpose for usage, if instructions were followed, the level of efficacy, the most used programs, the least used programs, and the like. The information can be used to improve the therapy and offer better user services. Based on the mined data, new therapies and products can be promoted to professionals if allowed by the user.

The disclosed methods provide software defined healthcare therapy, which is enabled by a more capable common therapy hardware and cloud platform. It creates the opportunity to deliver a greater number of therapies on the same piece of hardware, saving the cost and time. It eliminates the need to have professionals to perform the treatment. Free therapy programs allow users access and use free of charge; and paid therapy programs allow professionals and healthcare organizations to monetize their treatment solutions and IPs. The above recommendation algorithm can be further enhanced and weighed in a user's favorited ones and all users' favorited and highly rated ones learned from big data in the cloud platform.

While the inventive features have been particularly shown and described with reference to preferred embodiments thereof, it will be understood by those in the art that the foregoing and other changes may be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

What is claimed is:

1. A system architecture for software-defined programmable healthcare therapy, comprising:
a programmable healthcare therapy stored in a cloud;
an App on a smartphone device; and
a smart medical device,
wherein the App on the smartphone device can access the programmable healthcare therapy and perform the healthcare therapy through the smart medical device.

2. The system architecture of claim 1, further comprising a user account management module, a user data module, a healthcare therapy program encryption module, a management module, an operation management module, an App management module, a healthcare smart medical device firmware management module, a data mining module, or a service management module on the smartphone device.

3. The system architecture of claim 2, further comprising a synchronization with the cloud on the healthcare therapy program management module, an account information module, a user data module, a device firmware module on the smartphone device.

4. The system architecture of claim 1, further comprising a user interface provided by the App, which functions as a self-made healthcare therapy program, a healthcare therapy search and selection program, a healthcare therapy instruction program, a professional guide program, or a healthcare therapy operation and control program.

5. The system architecture of claim 1, wherein the smartphone App interacts with the smart medical device for link encryption and management, or healthcare therapy management and control.

6. A method of preparing a healthcare therapy program, comprising:
setting a building block of waveform parameters; and
combining the set of building block waveform parameters to form a healthcare therapy program.

7. The method of claim 6, further comprising:
testing and adjusting the healthcare therapy program;
adding usage instructions to the healthcare therapy program; and
uploading the healthcare therapy program to a cloud.

8. The method of claim 7, wherein a smartphone device App can then be notified of any new healthcare therapy programs or updates.

9. The method of claim 8, wherein the smartphone device App can periodically scan the cloud for any new programs or updates.

10. The method of claim 6, wherein the building block waveform parameters include shape type, frequency, symmetry, asymmetry, burst, interval, time or a combination thereof.

11. The method of claim 7, wherein a smartphone device App can request a download of one or more healthcare therapy programs from the cloud.

12. A method of preparing a healthcare therapy program on a smartphone device, comprising:
choosing therapy blocks for the healthcare therapy program;
associating time for use of the chosen therapy blocks for the healthcare therapy program; and
saving the prepared healthcare therapy program on an App on the smartphone device.

13. The method of claim 12, further comprising naming the saved healthcare therapy program.

14. The method of claim 13, further comprising saving the prepared healthcare therapy program in a cloud or on a smart medical device.

15. The method of claim 14, further comprising allowing access to the saved prepared healthcare therapy program on the App on the smartphone device, the cloud or the smart medical device.

16. The method of claim 12, further comprising adjusting an intensity of the healthcare therapy program on the App on the smartphone device or on a smart medical device.

17. A method of using one app with one RF transceiver to securely bind and maintain the binding with multiple device.

18. A method of using one app to control multiple therapy device through switching and re-synchronizing the operation and states.
